(19) United States
(12) Patent Application Publication
(10) Pub. No.: US 2026/0245852 A1
(43) Pub. Date: Aug. 20, 2026

(54) APPARATUS FOR AUTOMATICALLY MEASURING AND MONITORING TASTE AND ODOR-CAUSING SUBSTANCE IN WATER (RAW WATER, PURIFIED WATER, AND DISCHARGED WATER)

(71) Applicant: KEYSTONE SNT INC., Hanam-si (KR)

(72) Inventor: Sun Ki KIM, Seoul (KR)

(21) Appl. No.: 19/161,498

(22) PCT Filed: Nov. 8, 2024
(86) PCT No.: PCT/KR2024/017666

§ 371 Date: Sep. 2, 2025

(30) Foreign Application Priority Data
Jan. 26, 2024 (KR) 10-2024-0012152

(51) Int. Cl. H01J 49/04; H01J 49/00

(52) U.S. Cl. CPC H01J 49/0454; H01J 49/0031; H01J 49/0445

(57) ABSTRACT

The apparatus for automatically measuring and monitoring taste and odor-causing substances in water (raw water, purified water, and discharged water) according to an embodiment of the present invention includes: a vaporizing unit which introduces water (raw water, purified water, and discharged water), atomizes the water by using an ultrasonic vibrator, and then sprays the atomized water into a spray chamber, thereby generating an aerosol-type sample; an MS/MS mass spectrometry unit which applies a chemical ionization method using plasma and a quadrupole mass spectrometry (MS/MS) to the aerosol-type sample generated in the vaporization unit to measure mass values of 2-MIB S and Geosmin, which are taste and odor-causing substances; and a quantifying unit which quantifies the mass values of 2-MIB and Geosmin relative to the sample mass, measured by the MS/MS mass spectrometry unit.

### TECHNICAL FIELD

The disclosure relates to a device and method for automatically measuring and monitoring taste and odor causing substances in water (raw water, purified water, discharge water).

### BACKGROUND ART

Taste and odor causing substances related to water supply may be largely classified into natural and artificial ones, but those naturally occurring by biological action are common.

Taste and odor substances caused by natural causes are mostly found to be caused by algae. Representative taste and odor substances generated from algae include 2-MIB and geosmin.

Although 2-MIB and geosmin should be treated to below the olfactory detection concentration before entering the distribution water network by responding with rapid water purification treatment (ozone and hydrogen peroxide, powdered activated carbon injection, etc.) at a water treatment plant to avoid consumer complaints, currently at water treatment plants, the process involves extracting a sample, injecting the sample into a gas chromatograph with a separation column, thermally desorbing it, and analyzing it with a mass spectrometer. Since all analysis processes require a long analysis time due to manpower, immediate response is difficult, making it impossible to immediately respond when taste and odor substances occur, which limits the response to water purification treatment.

Accordingly, attempts are being made to build a system for automatically analyzing off-flavor substances [2-MIB, geosmin], but all processes involve manually collecting and analyzing samples, which requires a lot of measurement time and is not practical.

Therefore, there is a need to develop an economical and practical system that may measure off-flavor substances in real-time.

### PRIOR TECHNICAL DOCUMENTS

### Patent Documents

Korean Patent No. 10-0901779 (Jun. 2, 2009), “Internet-based Water Quality Measurement Monitoring Control System”
Korean Patent No. 10-0522764 (Oct. 12, 2005), “Real-time Water Quality Monitoring Device and Its Control Method”
Korean Patent No. 10-1406884 (Jun. 5, 2014), “Multi-wavelength Analysis-based Online Water Quality Measurement System for Real-time Detection of Organic Pollutants in Water Quality”
Korean Patent No. 10-1253251 (Apr. 4, 2013), “Real-time Taste and Odor Causing Substance Monitoring Control Device and Method for Water Purification Treatment”

### DETAILED DESCRIPTION OF THE INVENTION

### Technical Problem

An object of the present invention is to provide a device and method for automatically measuring and monitoring taste and odor causing substances in water (raw water, purified water, discharge water) using a new method.

### Technical Solution

To achieve the object, an device for automatically measuring and monitoring taste and odor causing substances in water according to an embodiment of the present invention includes a vaporization unit introducing raw water, purified water, or discharge water and atomizing the water through an ultrasonic vibrator to generate an aerosol-form sample, an MS/MS mass analysis unit applying a chemical ionization method using plasma and quadrupole mass spectrometry (MS/MS) to the aerosol-form sample generated in the vaporization unit to measure mass values of 2-MIB and geosmin which are taste and odor causing substances, and a quantification unit quantifying specific ion masses among fragment ion masses of 2-MIB and geosmin relative to sample mass measured by the MS/MS mass analysis unit.

In an embodiment, the vaporization unit includes a water tank unit having an inlet/outlet pipe formed therein for introducing and discharging the water (raw water, purified water, discharge water), an ultrasonic vibration unit atomizing the water introduced into the water tank unit through an ultrasonic vibrator, and a spray chamber generating the atomized water as an aerosol-form sample.

In an embodiment, the vaporization unit and the MS/MS mass analysis unit are connected by a Teflon tube.

In an embodiment, the Teflon tube is provided with a heating line for maintaining a surface temperature at a predetermined temperature for stabilization with atmospheric temperature.

In an embodiment, the MS/MS mass analysis unit continuously draws atmospheric moisture into an ion source and exposes the moisture to plasma to generate H₃O⁺ ions in plasma form generated by corona discharge, then induces ion reaction with the aerosol-form sample introduced from the vaporization unit to primarily calculate the ion masses of 2-MIB and geosmin which are sample substances.

In an embodiment, the MS/MS mass analysis unit secondarily calculates specific ion masses among fragment ion masses of 2-MIB and geosmin obtained by filtering other volatile organic compounds from the sample substances through collision cell reaction using air.

A method for automatically measuring and monitoring taste and odor causing substances in water (raw water, purified water, discharge water) according to an embodiment of the present invention for solving the problem includes a sample generation step of introducing raw water, purified water, or discharge water in a vaporization unit and atomizing the water through an ultrasonic vibrator to generate an aerosol-form sample, a step of applying a chemical ionization method using plasma and quadrupole mass spectrometry (MS/MS) to the aerosol-form sample generated in the vaporization unit in an MS/MS mass analysis unit to measure and monitor mass values of 2-MIB and geosmin which are taste and odor causing substances, and a step of quantifying specific ion masses among fragment ion masses of 2-MIB and geosmin relative to sample mass measured by the MS/MS mass analysis unit in a quantification unit.

In an embodiment, the sample measurement and monitoring step includes a step of continuously drawing atmospheric moisture into an ion source in the MS/MS mass analysis unit and exposing the moisture to plasma to generate H₃O⁺ ions in plasma form generated by corona discharge, then inducing ion reaction with the aerosol-form sample introduced from the vaporization unit to primarily calculate the ion masses of 2-MIB and geosmin which are sample substances, and a step of secondarily calculating specific ion masses among fragment ion masses of 2-MIB and geosmin obtained by filtering other volatile organic compounds from the sample substances through collision cell reaction using air.

### Advantageous Effects

Using the device and method for automatically measuring and monitoring taste and odor causing substances in water (raw water, purified water, discharge water) according to an embodiment of the present invention, unlike conventional analysis methods, columns or gas chromatograph devices for separating the substances are not needed, enabling measurement time reduction. Further, real-time measurement of taste and odor causing substances is possible during high water temperature algae occurrence periods, enabling cost reduction and budget savings through efficient injection of appropriate amounts of treatment chemicals (ozone, hydrogen peroxide, powdered activated carbon, etc.).

Further, by transmitting data to a control room in real-time, real-time monitoring of odor causing substance concentrations in raw water sources, which was difficult to monitor due to delayed surveillance of real-time changing concentrations with conventional analysis systems, is possible. Particularly, measurement is possible even during nights and weekends when people are not working, enabling rapid detection of the occurrence of taste and odor causing substances in water (raw water, purified water, discharge water), and accordingly, rapid response measures such as strengthening water purification treatment at water treatment plants may be taken to minimize damage such as tap water quality deterioration due to taste and odor substances.

### BRIEF DESCRIPTION OF DRAWINGS

FIG. 1 is a characteristic table of geosmin and 2-MIB.

FIG. 2 is an example view illustrating a device for automatically measuring and monitoring taste and odor causing substances in water (raw water, purified water, discharge water) according to an embodiment of the present invention.

FIG. 3 is a view illustrating arrangement changes of ultrasonic vibrator constituent materials according to the presence or absence of current application.

FIG. 4 is a detailed configuration diagram illustrating the MS/MS mass analysis unit illustrated in FIG. 1.

FIG. 5 is an example view for describing the stability region of a quadrupole.

FIG. 6 is an example view for describing a particle filtration method using a scan line.

FIG. 7 is a flowchart describing a method for automatically measuring and monitoring taste and odor causing substances in water (raw water, purified water, discharge water) according to an embodiment of the present invention.

### MODE FOR CARRYING OUT THE INVENTION

The terms as used herein are provided merely to describe some embodiments thereof, but not to limit the present disclosure. The terms as used herein are provided merely to describe some embodiments thereof, but not to limit the scope of other embodiments of the present disclosure. Unless otherwise defined, all terms including technical and scientific terms used herein have the same meaning as commonly understood by one of ordinary skill in the art to which the embodiments of the present invention pertain and should not be interpreted as overly broad or narrow. As used herein, terms wrong or inappropriate for representing the spirit of the present invention may be replaced with and understood as more proper ones to represent the spirit of the present invention by one of ordinary skill in the art. General terms as used herein should be interpreted in the context of the specification or as defined in dictionaries.

As used herein, the singular forms “a,” “an,” and “the” are intended to include the plural forms as well, unless the context clearly indicates otherwise. As used herein, the term “comprise,” “include,” or “have” should be appreciated not to preclude the presence or addability of features, numbers, steps, operations, components, parts, or combinations thereof as set forth herein.

The terms coming with ordinal numbers such as ‘first’ and ‘second’ may be used to denote various components, but the components are not limited by the terms. The terms are used only to distinguish one component from another. For example, a first component may be denoted a second component, and vice versa without departing from the scope of the present disclosure.

Hereinafter, exemplary embodiments of the inventive concept will be described in detail with reference to the accompanying drawings. The same reference denotations may be used to refer to the same or substantially the same elements throughout the specification and the drawings. No duplicate description of the same elements is given herein.

When determined to make the subject matter of the present invention unclear, the detailed description of the known art or functions may be skipped. The accompanying drawings are provided for an easier understanding of the spirit of the reception but the present invention should not be limited thereby.

Hereinafter, a device and method for automatically measuring and monitoring taste and odor causing substances in water (raw water, purified water, discharge water) according to an embodiment of the present invention is described in more detail based on the attached drawings.

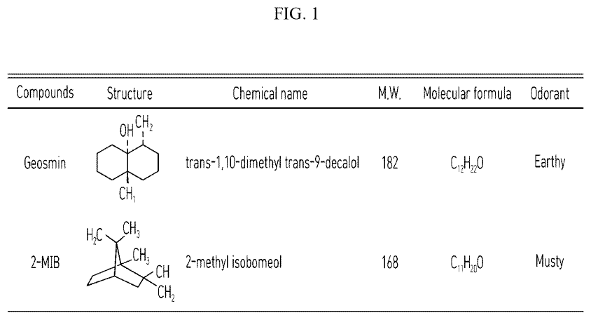

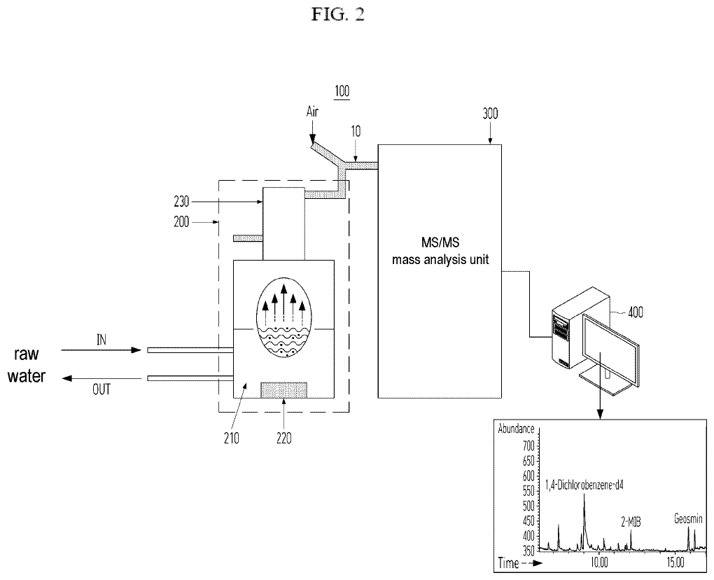

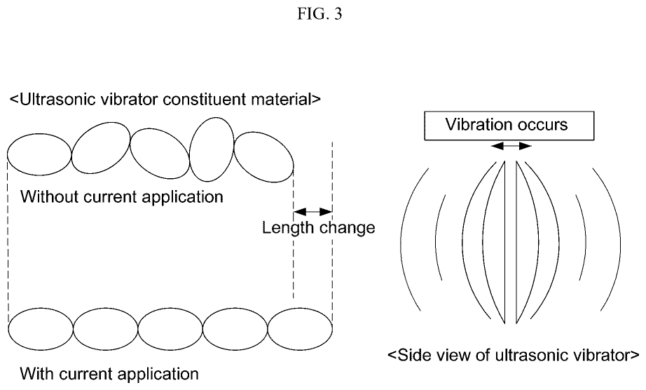

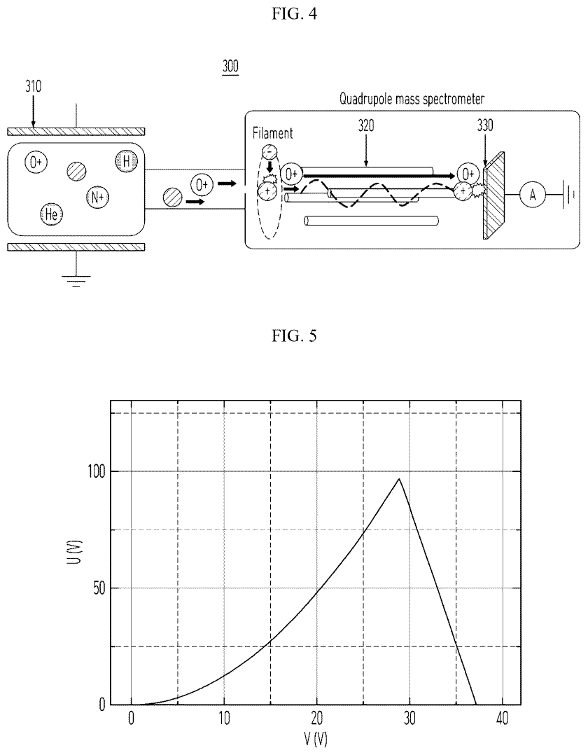

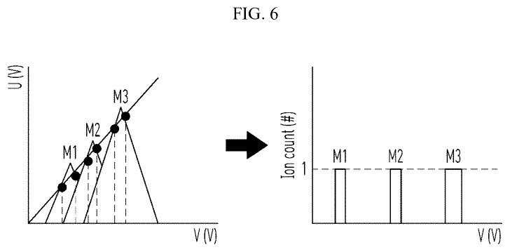

FIG. 1 is a characteristic table of geosmin and 2-MIB, FIG. 2 is an example view illustrating a device for automatically measuring and monitoring taste and odor causing substances in water (raw water, purified water, discharge water) according to an embodiment of the present invention, FIG. 3 is a view illustrating arrangement changes of ultrasonic vibrator constituent materials according to the presence or absence of current application, FIG. 4 is a detailed configuration diagram illustrating the MS/MS mass analysis unit illustrated in FIG. 1, FIG. 5 is an example view for describing the stability region of a quadrupole, and FIG. 6 is an example view for describing a particle filtration method using a scan line.

Meanwhile, before describing the present invention, the present invention is a device and method for measuring and monitoring ion masses of 2-MIB and geosmin which are taste and odor causing substances in water (raw water, purified water, discharge water). Unlike conventional analysis methods (Headspace-SPME-mass spectrometry), columns or gas chromatograph devices for separating the substances are not needed, measurement time may be shortened, and real-time measurement of taste and odor causing substances is possible during high water temperature algae occurrence periods, enabling cost reduction and budget savings through efficient injection of appropriate amounts of treatment chemicals (ozone, hydrogen peroxide, powdered activated carbon, etc.), which may be a new concept device and method.

As illustrated in FIG. 1, a device 100 for automatically measuring and monitoring taste and odor causing substances in water (raw water, purified water, discharge water) according to an embodiment of the present invention includes a vaporization unit 200, an MS/MS mass analysis unit 300, and a quantification unit 400.

The vaporization unit 200 may be a component for introducing raw water, purified water, or discharge water and atomizing the water through an ultrasonic vibrator to generate an aerosol-form sample.

More specifically, the vaporization unit 200 includes a water tank unit 210, an ultrasonic vibration unit 220, and a spray chamber 230.

The water tank unit 210 is a component for introducing and discharging water (raw water, purified water, discharge water), and may be a glass tube having an inlet/outlet pipe formed therein.

The ultrasonic vibration unit 220 may be a component for atomizing the raw water introduced into the water tank unit 210 through an ultrasonic vibrator.

The spray chamber 230 may be a component for collecting the atomized water (raw water, purified water, discharge water) as the aerosol-form sample and then spraying it to a Teflon tube 10 described below.

The aerosol-form sample collected in the spray chamber 230 is delivered to an MS/MS mass analysis unit 300 described below along the Teflon tube 10.

The Teflon tube 10 may be provided with a heating line for maintaining a surface temperature at a predetermined temperature to prevent the moving aerosol-form sample from liquefying due to atmospheric temperature differences.

Next, the MS/MS mass analysis unit 300 may be a component for applying a chemical ionization method using plasma and quadrupole mass spectrometry (MS/MS) to the aerosol-form sample generated in the vaporization unit 200 to measure mass values of 2-MIB and geosmin which are taste and odor causing substances.

The MS/MS mass analysis unit 300 may be a component for continuously drawing atmospheric moisture into an ion source and exposing the moisture to plasma to generate H₃O⁺ ions in plasma form generated by corona discharge, then inducing ion reaction with the aerosol-form sample introduced from the vaporization unit to primarily calculate the ion masses of 2-MIB and geosmin which are sample substances.

The MS/MS mass analysis unit 300 may be a component for secondarily calculating specific ion masses among fragment ion masses of 2-MIB and geosmin obtained by filtering other organic compounds from the sample substances through collision cell reaction using air.

The MS/MS mass analysis unit 300 may be a component for connecting two MS (Mass Spectrometry) units in parallel to detect ionized substances according to mass to charge ratio.

More specifically, the MS/MS mass analysis unit 300 includes an ionization unit 310, a quadrupole 320, and a mass detection unit 330.

The ionization unit 310 may be a component for continuously drawing atmospheric moisture into an ion source and exposing the moisture to plasma to generate H₃O⁺ ions in plasma form generated by corona discharge.

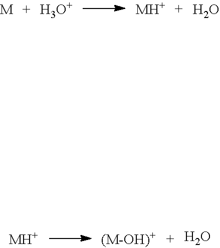

Further, it may be a component for generating ion reaction of the aerosol-form sample collected in the vaporization unit 200.

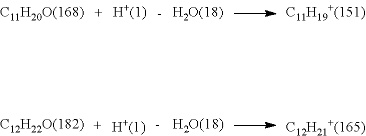

The ionization unit 310 may be operated by any one of a thermoelectron method using a filament, a laser method, and an inductively coupled plasma method according to the driving method, and in the present invention, it is described as an inductively coupled plasma method.

Next, the quadrupole 320 may be a component for filtering particles to be measured by applying DC power and AC power to two pairs of conductor rods.

When DC and AC power are applied to the quadrupole, an electric field is determined within the quadrupole, and incident ions are accelerated by this electric field and move.

Among these, particles that move stably and reach the ammeter are measured, while particles that move unstably and collide with the quadrupole are not measured, which is the principle of measuring only the particles we want.

A sample ionized in the ionization unit 310 enters the inside of the quadrupole, and in this case, only particles of specific mass that may pass under the DC and AC voltages applied to the quadrupole pass through the quadrupole, while particles with other masses collide with the quadrupole and are destroyed.

For reference, in the case of an ideal quadrupole filter, hyperbolic electrodes should be disposed in a quadrupole form, and the Laplace equation for the potential inside the quadrupole is known as follows.

$${∇}^{2} V = α {x}^{2} + β {y}^{2} + γ {z}^{2} \qquad [ Equation 1 ]$$

Here, α, β, and γ are values according to the positions of x, y, and z, respectively. The voltages applied to the two electrodes positioned on the x-axis and the two electrodes positioned on the y-axis are as illustrated in Equations 2 and 3 below.

$${ϕ}_{x} = U + V cos ( ω t ) \qquad [ Equation 2 ]$$

$${ϕ}_{y} = - \{ U + V cos ( ω t ) \} \qquad [ Equation 3 ]$$

Here, U means DC voltage and V means the magnitude of RF voltage. To obtain the equation of motion of ions passing through the quadrupole filter, the potential within the quadrupole filter should first be obtained. The potential within the quadrupole should satisfy the Laplace equation and there is no change along the z-axis. Therefore, the potential is as illustrated in Equation 4 below.

$${ϕ}_{x y} = \frac{{ϕ}_{0}}{2 {r}_{0}} ( {x}^{2} - {y}^{2} ) \qquad [ Equation 4 ]$$

Here, r₀ means the distance from the origin to one cylinder of the quadrupole, and φ₀ means the DC and AC voltages applied to the quadrupole. By putting the potential into the Lorentz equation, the following equation of motion of ions may be derived as illustrated in Equation 5.

$$\frac{{d}^{2} u}{d {ξ}^{2}} + ( {a}_{u} - 2 {q}_{u} cos 2 ξ ) u = 0 \qquad [ Equation 5 ]$$

Here, ξ=ωt, ω=2πf. au and qu are called stability factors and their values are as illustrated in Equations 6 and 7, respectively.

$${a}_{u} = {a}_{x} = - {a}_{y} = 8 ZeU / {mr}_{0}^{2} {ω}^{2} \qquad [ Equation 6 ]$$

$${q}_{u} = {q}_{x} = - {q}_{y} = 4 ZeU / {mr}_{0}^{2} {ω}^{2} \qquad [ Equation 7 ]$$

Z represents the charge amount of the ion and e represents the elementary charge amount. The equation of motion is called the Mathieu equation.

Using the Mathieu equation, if only the voltage conditions for particles to pass through without hitting the quadrupoles positioned on the x and y axes are drawn, a triangular region as illustrated in FIG. 4 may be obtained.

This is called the stability region for the particle, and since this stability region is the transmission status when particles of specific conditions are incident to the quadrupole, it may be said to be the transmission characteristics when the particle enters the quadrupole filter.

Based on this stability region, the voltage conditions on the left are conditions for hitting the rod positioned on the y-axis, and the right are conditions for hitting the rod positioned on the x-axis.

Further, even when the condition is to hit the x-axis or y-axis rod, a condition closer to the stability region has the characteristic of hitting the quadrupole later compared to a condition positioned farther away.

Meanwhile, the DC and AC power applied to the quadrupole is usually represented as follows.

$$U = 1.1 \; 67 V$$

Here, U means DC voltage and V means AC voltage.

A voltage application method in which AC voltage and DC voltage are represented in a linear function form is called a scan line.

A particle filtration method using a scan line may be represented as illustrated in FIG. 6.

In the region where the scan line and the stability region of incident particles meet, particles pass through the quadrupole, and in places where they do not meet, they are filtered.

Therefore, when different particles are incident, since the sizes of their respective stability regions are different, when voltage is applied along the scan line, the transmission status of each particle according to the applied AC voltage may be known as illustrated on the right side of FIG. 6.

The sample filtered in this way hits the mass detection unit 330 to generate current, and by measuring this current, the type of gas sample and the mass to charge ratio (m/z) may be known.

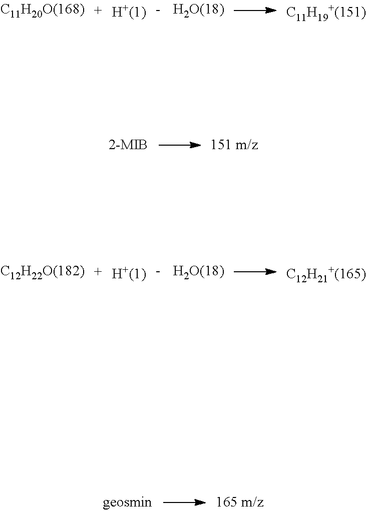

The mass detection unit 330 usually uses a Faraday cup or an electron multiplier.

Meanwhile, since there may be other organic compounds besides sample substances in the sample to be measured, the MS/MS mass analysis unit 300 proceeds with collision cell reaction once more to conduct measurement with the following final mass values.

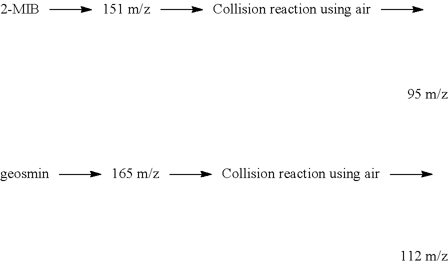

The quantification unit 400 may be a component for quantifying specific ion masses among fragment ion masses of 2-MIB and geosmin relative to sample mass measured by the MS/MS mass analysis unit 300.

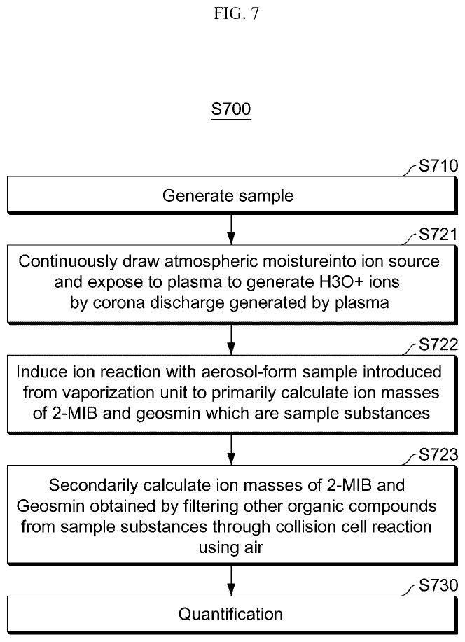

FIG. 7 is a flowchart describing a method for automatically measuring and monitoring taste and odor causing substances in water (raw water, purified water, discharge water) according to an embodiment of the present invention.

Referring to FIG. 7, a method S700 for automatically measuring and monitoring taste and odor causing substances in water (raw water, purified water, discharge water) according to an embodiment of the present invention includes a sample generation step S710, a sample measurement and monitoring step S720, and a quantification step S730.

The sample generation step S710 may be a step of introducing raw water, purified water, or discharge water in a vaporization unit and atomizing the water through an ultrasonic vibrator to generate an aerosol-form sample.

The sample measurement and monitoring step S720 may be a step of applying a chemical ionization method using plasma and quadrupole mass spectrometry (MS/MS) to the aerosol-form sample generated in the vaporization unit in an MS/MS mass analysis unit to measure mass values of 2-MIB and geosmin which are taste and odor causing substances.

The sample measurement and monitoring step S720 includes a process of generating (S721) H₃O₊ ions in plasma form generated by corona discharge by continuously drawing atmospheric moisture into an ion source in the MS/MS mass analysis unit and exposing the moisture to plasma, then inducing ion reaction with the aerosol-form sample introduced from the vaporization unit to primarily calculate (S722) the ion masses of 2-MIB and geosmin which are sample substances, and secondarily calculating (S723) specific ion masses among fragment ion masses of 2-MIB and geosmin obtained by filtering other organic compounds from the sample substances through collision cell reaction using air.

Next, the quantification step S730 may be a step S730 of quantifying specific ion masses among fragment ion masses of 2-MIB and geosmin relative to sample mass measured by the MS/MS mass analysis unit in a quantification unit.

Therefore, using the device and method for automatically measuring and monitoring taste and odor causing substances in water (raw water, purified water, discharge water) according to an embodiment of the present invention, unlike conventional analysis methods, columns or gas chromatograph devices for separating the substances are not needed, thereby shortening measurement time. Further, real-time measurement of taste and odor causing substances is possible during high water temperature algae occurrence periods, enabling cost reduction and budget savings through efficient injection of appropriate amounts of treatment chemicals (ozone, hydrogen peroxide, powdered activated carbon, etc.).

Further, by transmitting data to a control room in real-time, real-time monitoring of odor causing substance concentrations in raw water sources, which was difficult to monitor in real-time with conventional analysis systems due to delayed surveillance of real-time changing concentrations, is possible. Particularly, measurement is possible even during nights and weekends when people are not working, enabling rapid detection of the occurrence of taste and odor causing substances in water (raw water, purified water, discharge water), and accordingly, rapid response measures such as strengthening water purification treatment at water treatment plants may be taken to minimize damage such as tap water quality deterioration due to taste and odor substances.

The above-described devices may be implemented as hardware components, software components, and/or in a combination thereof. For example, the devices and components described herein may be implemented using one or more general-purpose or specific-purpose computers, such as processors, controllers, arithmetic logic units (ALUs), digital signal processors, micro-computers, field programmable arrays (FPAs), programmable logic units (PLUs), micro-processors, any other devices capable of executing and responding to instructions.

The processing device or processor may perform an operating system (OS) and one or more software applications performed on the OS. The processing device or processor may access, store, manipulate or control, process, and generate data in response to the execution of the software. For illustration purposes, the processing device or processor may be a single one but it will be appreciated by one of ordinary skill in the art that a processing device may include a plurality of processing elements and/or a plurality of types of processing elements.

For example, the processing device may include a plurality of processors or a single processor and a single controller. The server or device may have other various processing configurations, such as parallel processors. The software may include computer programs, codes, instructions, or combinations of one or more thereof and may configure the processing device as it is operated as desired or may instruct the processing device independently or collectively. The software and/or data may be embodied permanently or temporarily in signal waves transmitted or computer storage media or devices, virtual equipment, physical devices, components, or any type of machine so as to provide instructions or data to the processing device or to be interpreted by the processing device. The software may be distributed over computer systems connected together via a network to be distributively stored or executed. The software and data may be stored in one or more computer readable recording media.

The methods according to the embodiments may be implemented in the form of programming commands executable by various computer means, and the programming commands may be recorded in a computer-readable medium. The computer-readable medium may include programming commands, data files, or data structures, alone or in combinations thereof. The programming commands recorded in the medium may be specially designed and configured for the present invention or may be known and available to one of ordinary skill in the computer software-related art. Examples of the computer readable recording medium may include, but is not limited to, magnetic media, such as hard disks, floppy disks or magnetic tapes, optical media, such as CD-ROMs or DVDs, magneto-optical media, such as floptical disks, memories, such as ROMs, RAMs, or flash memories, or other hardware devices specially configured to retain and execute programming commands. Examples of the programming commands may include, but are not limited to, high-level language codes executable by a computer using, e.g., an interpreter, as well as machine language codes as created by a compiler. The above-described hardware devices may be configured to operate as one or more software modules to perform operations according to an embodiment of the present invention, or the software modules may be configured to operate as one or more hardware modules to perform the operations.

Although the disclosure is shown and described in connection with embodiments, it will be easily appreciated by one of ordinary skill in the art that various changes or modifications may be made without departing from the scope of the disclosure. For example, although the techniques described herein are performed in a different order from those described herein and/or the components of the above-described structure or device are coupled, combined, or assembled in a different form from those described herein, or some components are replaced with other components or equivalents thereof, a proper result may be achieved. Hence, other implementations, other embodiments, and equivalents to the claims also belong to the scope of the claims described below.

### LEGEND OF REFERENCE NUMBERS

100: Device for automatically measuring and monitoring taste and odor causing substances in water (raw water, purified water, discharge water)
200: Vaporization unit
210: Ultrasonic vibrator humidification unit
220: Spray chamber
300: MS/MS mass analysis unit
310: Ionization unit
320: Quadrupole
330: Ion mass detection unit
400: Quantification unit

1. An device for automatically measuring and monitoring taste and odor causing substances in water (raw water, purified water, discharge water), comprising:
a vaporization unit introducing raw water, purified water, or discharge water and atomizing the water through an ultrasonic vibrator to generate an aerosol-form sample;
a quadrupole mass spectrometry (MS/MS) mass analysis unit applying a chemical ionization method using plasma and quadrupole mass spectrometry (MS/MS) to the aerosol-form sample generated in the vaporization unit to measure mass values of 2-MIB and geosmin which are taste and odor causing substances; and
a quantification unit quantifying ion masses of 2-MIB and geosmin relative to sample mass measured by the MS/MS mass analysis unit.

2. The device of claim 1, wherein the vaporization unit includes:
a water tank unit (10) having an inlet/outlet pipe (11) formed therein for introducing and discharging the water (raw water, purified water, discharge water);
an ultrasonic vibration unit (20) atomizing the water (raw water, purified water, discharge water) introduced into the water tank unit (10) through the ultrasonic vibrator; and
a spray chamber (30) generating the atomized water (raw water, purified water, discharge water) as the aerosol-form sample.

3. The device of claim 1, wherein the vaporization unit and the MS/MS mass analysis unit are connected by a Teflon tube.

4. The device of claim 3, wherein the Teflon tube is provided with a heating line for maintaining a surface temperature at a predetermined temperature for stabilization with atmospheric temperature.

5. The device of claim 1, wherein the MS/MS mass analysis unit continuously draws atmospheric moisture into an ion source and exposes the moisture to plasma to generate H₃O⁺ ions by corona discharge generated by the plasma, then induces ion reaction with the aerosol-form sample introduced from the vaporization unit to primarily calculate the ion masses of 2-MIB and geosmin which are sample substances.

6. The device of claim 1, wherein the MS/MS mass analysis unit secondarily calculates the ion masses of 2-MIB and geosmin obtained by filtering other organic compounds from the sample substances through collision cell reaction using air.

7. A method for automatically measuring and monitoring taste and odor causing substances in water (raw water, purified water, discharge water), comprising:
a sample generation step of introducing raw water, purified water, or discharge water in a vaporization unit and atomizing the water through an ultrasonic vibrator to generate an aerosol-form sample;
a sample measurement and monitoring step of applying a chemical ionization method using plasma and quadrupole mass spectrometry (MS/MS) to the aerosol-form sample generated in the vaporization unit in an MS/MS mass analysis unit to measure mass values of 2-MIB and geosmin which are taste and odor causing substances; and
a step of quantifying specific ion masses among fragment ion masses of 2-MIB and geosmin relative to sample mass measured by the MS/MS mass analysis unit in a quantification unit.

8. The device of claim 7, wherein the sample measurement and monitoring step includes:
a step of continuously drawing atmospheric moisture into an ion source in the MS/MS mass analysis unit and exposing the moisture to plasma to generate H₃O⁺ ions in plasma form generated by corona discharge, then inducing ion reaction with the aerosol-form sample introduced from the vaporization unit to primarily calculate the ion masses of 2-MIB and geosmin which are sample substances; and
a step of secondarily calculating specific ion masses among fragment ion masses of 2-MIB and geosmin obtained by filtering other organic compounds from the sample substances through collision cell reaction using air.
